(19) United States
(12) Patent Application Publication
(10) Pub. No.: US 2017/0290648 A1
(43) Pub. Date: Oct. 12, 2017

(54) LIGHT-GUIDING MOUTHGUARD ASSEMBLY

(71) Applicant: Choice Biotech Inc., Tainan City (TW)

(72) Inventor: Tai-Hong Kuo, Tainan City (TW)

(21) Appl. No.: 15/470,224

(22) Filed: Mar. 27, 2017

(30) Foreign Application Priority Data
Apr. 8, 2016 (TW) 105111039

(51) Int. Cl. A61C 19/06; A63B 71/08; A61M 16/04

(52) U.S. Cl. CPC A61C 19/06; A61M 16/049; A63B 71/085; A61M 16/0488; A61N 5/00

(57) ABSTRACT

A light-guiding mouthguard assembly has a light-transmittable mouthguard and two light-guiding strips. The light-transmittable mouthguard has a teeth-containing channel formed along a curve as a teeth arch. The two light-guiding strips are located on opposite sides of the teeth-containing channel respectively. Each one of the two light-guiding strips is mounted through two opposite terminals of the light-transmittable mouthguard along the teeth-containing channel. An exterior surface of each one of the two light-guiding strips is light-transmittable.

### CROSS-REFERENCE TO RELATED APPLICATIONS

This application claims the benefit of Taiwan patent application No. 105111039, filed on Apr. 8, 2016, the disclosure of which is incorporated herein in its entirety by reference.

### BACKGROUND OF THE INVENTION

### 1. Field of the Invention

The present invention relates to a mouthguard assembly, and more particularly to a light-guiding mouthguard assembly.

### 2. Description of Related Art

A person who cares about oral hygiene may brush the teeth routinely and have regular dental check-ups for teeth health care, such that the person may be less susceptible to dental diseases. On the contrary, when a person does not care about oral hygiene, the mouth of such person may be a hotbed of germs. For example, if the mouth cavity remains unclean for a long time, the germs in the mouth cavity will easily induce mouth illness, such as tooth decay or gum disease. When a tooth is severely decayed, the decayed tooth should be extracted or further treated with a surgery of root canal procedure. In addition, when the person's gum disease is severe, the person may have to undergo surgery treatments, laser treatments, or drug (e.g. antibiotics) treatments. Therefore, the person may have huge burdens both in mental stress and medical expenses.

To sterilize the mouth cavity, including the teeth, is an effective way for preventing mouth diseases or improving the oral hygiene. Photodynamic therapy (PDT) is a conventional way to sterilize the mouth cavity. The dentist may coat dye over inflammation cells in the mouth of the patient such that the dye may adhere to germs. Then the dentist inserts a handheld laser apparatus into the mouth of the patient. The laser apparatus may irradiate the dye in a particular light wavelength for duration, such as 10 seconds. As a result, the dye is excited to change to a triple state from a singlet state and form an oxygen free radical in a singlet state. The oxygen free radical may destroy the lipid and the enzyme of the germs to achieve the purpose of sterilization.

However, because the laser apparatus has to enter the mouth cavity of the patient, the movement of the laser apparatus may discomfort the patient. Besides, the movement range of the laser apparatus is limited such that the laser apparatus may fail to irradiate some areas of the mouth cavity. Hence, the laser apparatus fails to completely sterilize the mouth cavity.

### SUMMARY OF THE INVENTION

An objective of the present invention is to provide a light-guiding mouthguard assembly. A user may directly wear the light-guiding mouthguard assembly of the present invention in the mouth.

The light-guiding mouthguard assembly of the present invention comprises a light-transmittable mouthguard and two light-guiding strips. The light-transmittable mouthguard has a teeth-containing channel formed along a curve as a teeth arch. The two light-guiding strips are located on opposite sides of the teeth-containing channel respectively. Each one of the two light-guiding strips is continuous and uninterrupted and mounted through two opposite terminals of the light-transmittable mouthguard along the teeth-containing channel. An exterior surface of each one of the two light-guiding strips is light-transmittable.

The light-guiding mouthguard assembly of the present invention may be selectively mounted on the upper teeth and the lower teeth of the user. Because the teeth-containing channel is formed along the curve of the teeth arch, the user may directly wear the light-guiding mouthguard assembly of the present invention on the upper teeth or the lower teeth, such that the upper teeth or the lower teeth fit in the teeth-containing channel. The two light-guiding strips are mounted through the light-transmittable mouthguard along the teeth-containing channel. Hence, the light outputted from the two light-guiding strips may completely irradiate the mouth cavity, including the mouth tissues and the upper teeth or the lower teeth without any blind spot. In cooperation with the dye coated on the mouth tissues and the teeth, the dye would be excited by the light from the light-guiding strips to completely sterilize the germs in the mouth cavity. Especially, a sterilization depth may reach 6 mm by coating far infrared dye.

Another light-guiding mouthguard assembly of the present invention comprises a light-transmittable mouthguard, two upper light-guiding strips, and two lower light-guiding strips. The light-transmittable mouthguard has an upper teeth-containing channel and a lower teeth-containing channel that are formed along curves as an upper teeth arch and a lower teeth arch respectively. The two upper light-guiding strips are located on opposite sides of the upper teeth-containing channel respectively. Each one of the two upper light-guiding strips is continuous and uninterrupted and mounted through two opposite terminals of the light-transmittable mouthguard along the upper teeth-containing channel. An exterior surface of each one of the two upper light-guiding strips is light-transmittable. The two lower light-guiding strips are located on opposite sides of the lower teeth-containing channel respectively. Each one of the two lower light-guiding strips is continuous and uninterrupted and mounted through the two opposite terminals of the light-transmittable mouthguard along the lower teeth-containing channel. An exterior surface of each one of the two lower light-guiding strips is light-transmittable.

The user may directly wear the light-guiding mouthguard assembly of the present invention, such that the upper teeth and the lower teeth fit in the upper teeth-containing channel and the lower teeth-containing channel respectively as a same time. The upper light-guiding strips and the lower light-guiding strips are mounted through the light-transmittable mouthguard along the upper teeth-containing channel and the lower teeth-containing channel respectively. Hence, the light outputted from the upper light-guiding strips and the lower light-guiding strips may completely irradiate the mouth cavity, including the mouth tissues, the upper teeth, and the lower teeth without any blind spot. In cooperation with the dye coated on the mouth tissues and the teeth, the dye would be excited by the light from the upper light-guiding strips and the lower light-guiding strips to completely sterilize the germs in the mouth cavity.

The light-guiding mouthguard assembly of the present invention has simple structure and is easily applicable in practice. Compared with the conventional laser apparatus, the light-guiding mouthguard assembly of the present invention would not be moved in the user's mouth cavity and thus does not discomfort the user during a period of sterilization.

### BRIEF DESCRIPTION OF THE DRAWINGS

FIG. 1 is a perspective view of a first embodiment of the light-guiding mouthguard assembly of the present invention;

FIG. 2 is a sectional view of the first embodiment of the light-guiding mouthguard assembly of the present invention;

FIG. 3 is a top view of the first embodiment of the light-guiding mouthguard assembly of the present invention;

FIG. 4 is a perspective view of a second embodiment of the light-guiding mouthguard assembly of the present invention; and

FIG. 5 is a sectional view of the second embodiment of the light-guiding mouthguard assembly of the present invention.

### DETAILED DESCRIPTION OF THE PREFERRED EMBODIMENT

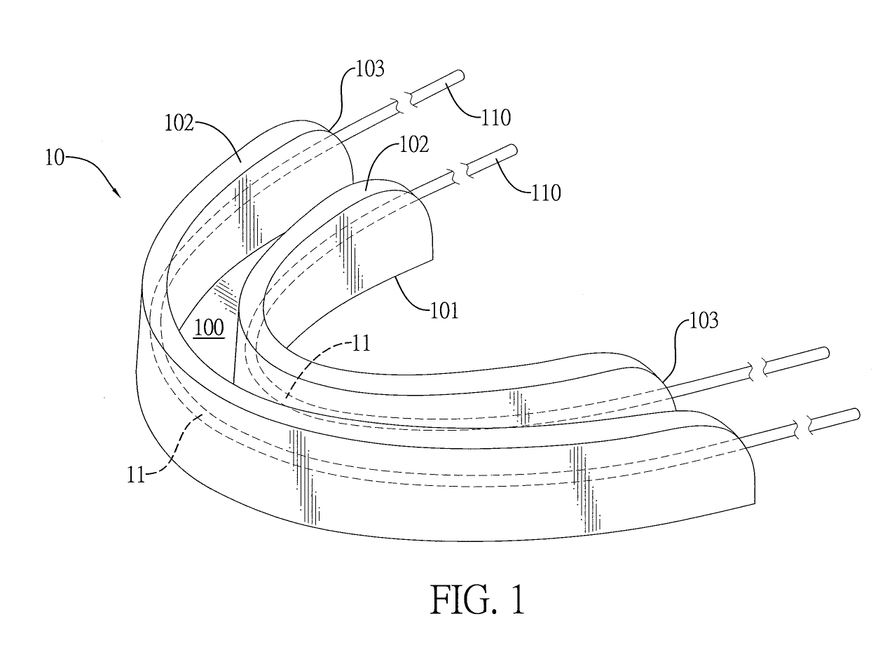

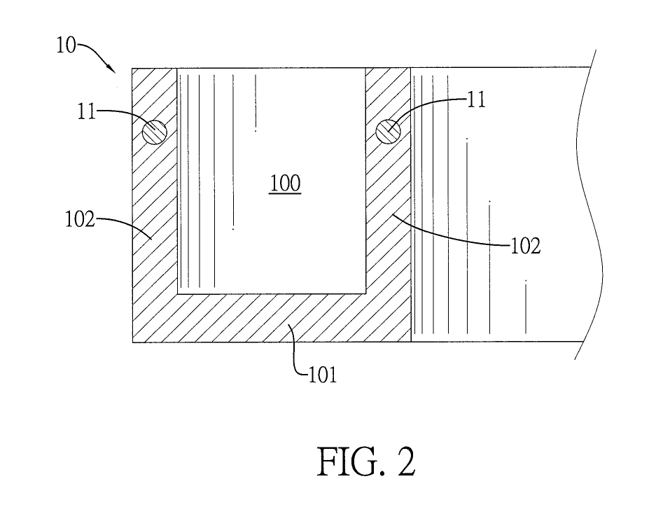

With reference to FIGS. 1 to 3, a first embodiment of the light-guiding mouthguard assembly of the present invention comprises a light-transmittable mouthguard 10 and two light-guiding strips 11.

The light-transmittable mouthguard 10 has a teeth-containing channel 100 formed along a curve as a teeth arch of a user, such as a patient. The teeth-containing channel 100 fits the contour of the upper teeth arch or the lower teeth arch of the user. When the user wears the light-transmittable mouthguard 10, the teeth-containing channel 100 may contain the upper teeth or the lower teeth of the user. In the first embodiment of the present invention, the light-transmittable mouthguard 10 may be an integrally formed mouthguard that comprises a substrate 101 and two light-transmittable walls 102. The substrate 101 is formed along the curve of the teeth arch. The two light-transmittable walls 102 are formed on a top surface of the substrate 101 along the curve of the teeth arch and opposite to each other. The teeth-containing channel 100 is formed within the substrate 101 and the two light-transmittable walls 102. For example, when the user wears the light-transmittable mouthguard 10 on the upper teeth, the two light-transmittable walls 102 are located on an inner side and an outer side of the upper teeth respectively.

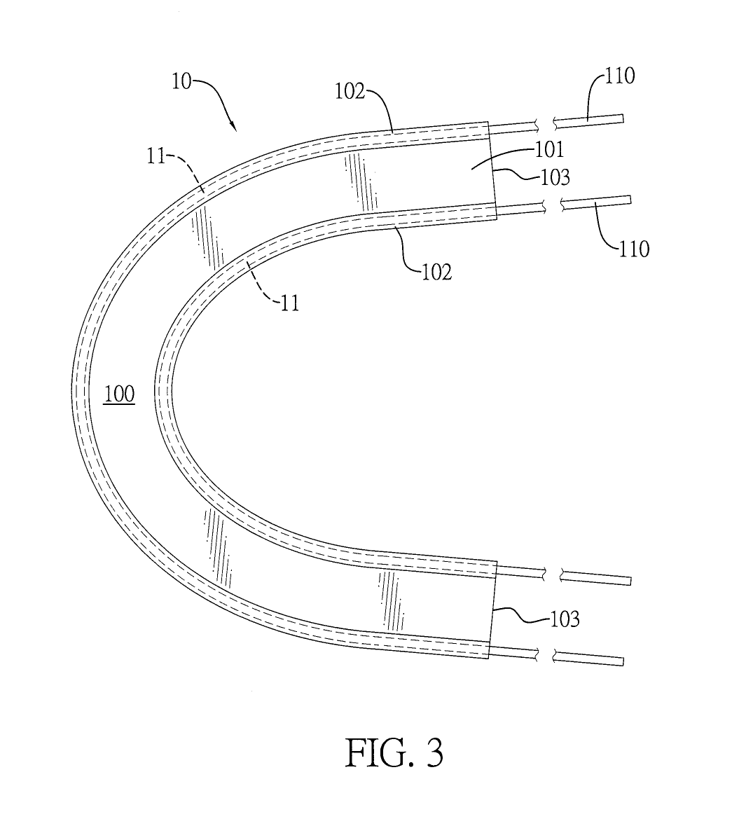

The two light-guiding strips 11 are located on opposite sides of the teeth-containing channel 100 respectively. As shown in FIG. 1 and FIG. 3, each one of the two light-guiding strips 11 is continuous and uninterrupted. Each one of the two light-guiding strips 11 is mounted through the light-transmittable mouthguard 10 along the teeth-containing channel 100, wherein each one of the light-guiding strips 11 has two ends 110 respectively extending through and exposed from two opposite terminals 103 of the light-transmittable mouthguard 10. In the first embodiment of the present invention, the two light-guiding strips 11 are mounted through the two light-transmittable walls 102 respectively. For example, when the user wears the light-guiding mouthguard assembly of the present invention on the upper teeth, the two light-transmittable walls 102 are located on the inner side and the outer side of the upper teeth respectively. An exterior surface of each one of the two light-guiding strips 11 is light-transmittable. For example, each one of the two light-guiding strips 11 may be a continuous and uninterrupted optical fiber strips. When a lighting device (not shown in the figure) emits light to the ends 110 of the light-guiding strips 11, the light emitted from the lighting device passes along the light-guiding strips 11 and is transmitted through the exterior surfaces of the light-guiding strips 11. For example, when the user wears the light-guiding mouthguard assembly of the present invention on the upper teeth, the light transmitted from the exterior surfaces of the light-guiding strips 11 irradiates the inner surfaces and the outer surfaces of the teeth and the mouth tissues, such as the gums.

The mouthguard assembly of the present invention may be applied to sterilize the teeth. In practice, the teeth and the mouth tissues of the user may be coated by dye. The light emitted from the lighting device may be infrared rays or far infrared rays. When the infrared rays or the far infrared rays irradiate the dye, the dye is excited to kill the germs, such as periodontal disease germs, and adheres to the teeth and the mouth tissues.

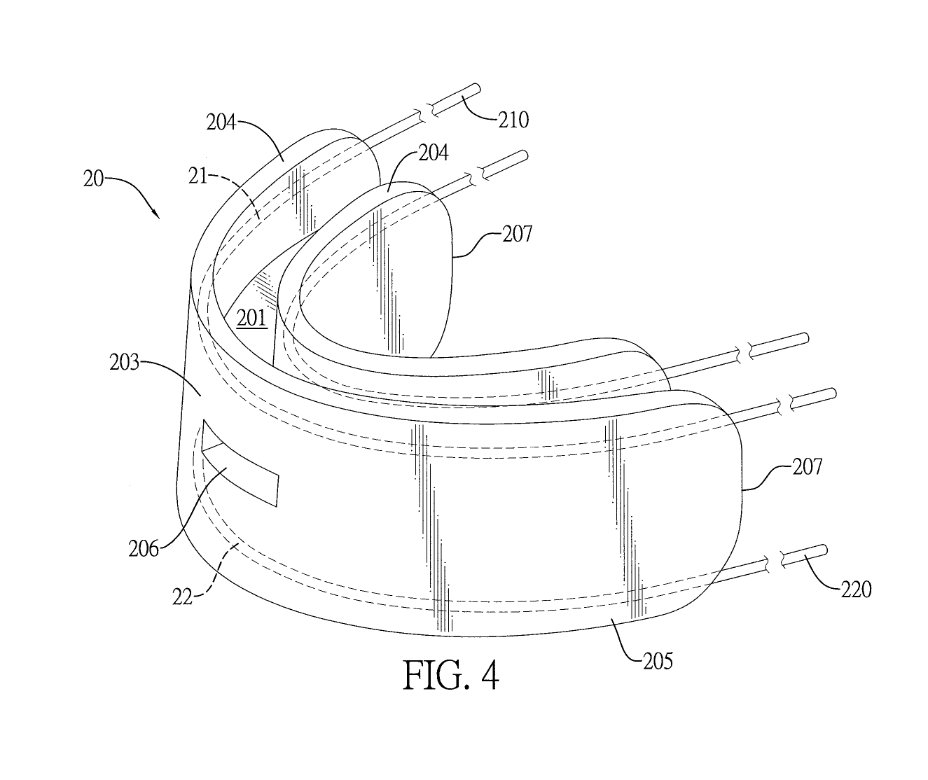

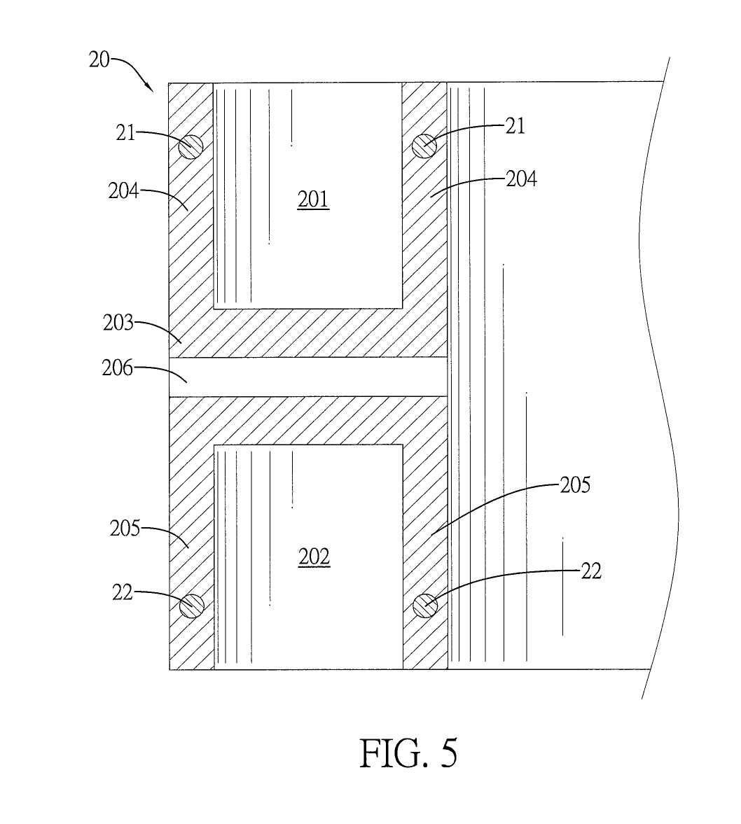

As mentioned above, the user may wear the first embodiment of the present invention with one single teeth-containing channel 100 in the mouth cavity to contain the upper teeth or the lower teeth by demand. With reference to FIGS. 4 and 5, a second embodiment of the present invention may contain the upper teeth and the lower teeth at a same time. The second embodiment of the light-guiding mouthguard assembly of the present invention comprises a light-transmittable mouthguard 20, two upper light-guiding strips 21, and two lower light-guiding strips 22. The upper light-guiding strips 21 and the lower light-guiding strips 22 are the same as the light-guiding strips 11 in the first embodiment, such that they are not repeatedly introduced herein. Exterior surfaces of the two upper light-guiding strips 21 and the two lower light-guiding strips 22 are light-transmittable.

The light-transmittable mouthguard 20 has an upper teeth-containing channel 201 and a lower teeth-containing channel 202 that are formed along curves as an upper teeth arch and a lower teeth arch of a user respectively. The upper teeth-containing channel 201 and the lower teeth-containing channel 202 fit the contour of the upper teeth arch and the lower teeth arch of the user respectively. Hence, when the user wears the light-transmittable mouthguard 20, the upper teeth-containing channel 201 may contain the upper teeth and the lower teeth-containing channel 202 may contain the lower teeth of the user. In the second embodiment, the light-transmittable mouthguard 20 may be an integrally formed mouthguard that comprises a substrate 203, two upper light-transmittable walls 204, and two lower light-transmittable walls 205.

The substrate 203 is formed along the curves of the upper teeth arch and the lower teeth arch of the user and has a top surface and a bottom surface. The two upper light-transmittable walls 204 are formed on the top surface of the substrate 203 along the curve of the upper teeth arch and opposite to each other. The upper teeth-containing channel 201 is formed within the substrate 203 and the two upper light-transmittable walls 204. The two lower light-transmittable walls 205 are formed on the bottom surface of the substrate 203 along the curve of the lower teeth arch and opposite to each other. The lower teeth-containing channel 202 is formed within the substrate 203 and the two lower light-transmittable walls 205.

The two upper light-guiding strips 21 are located on opposite sides of the upper teeth-containing channel 201 respectively. As shown in FIG. 4, each one of the two upper light-guiding strips 21 is continuous and uninterrupted. Each one of the two upper light-guiding strips 21 is mounted through the light-transmittable mouthguard 20 along the upper teeth-containing channel 201, wherein each one of the upper light-guiding strips 21 has two ends 210 respectively extending through and exposed from two opposite terminals 207 of the light-transmittable mouthguard 20. In the second embodiment, the two upper light-guiding strips 21 are mounted through the two upper light-transmittable walls 204 respectively.

The two lower light-guiding strips 22 are located on opposite sides of the lower teeth-containing channel 202 respectively. As shown in FIG. 4, each one of the two lower light-guiding strips 22 is continuous and uninterrupted. Each one of the two lower light-guiding strips 22 is mounted through the light-transmittable mouthguard 20 along the lower teeth-containing channel 202, wherein each one of the lower light-guiding strips 22 has two ends 220 respectively extending through and exposed from the two opposite terminals 207 of the light-transmittable mouthguard 20. In the second embodiment, the two lower light-guiding strips 22 are mounted through the two lower light-transmittable walls 205 respectively.

When the user wears the light-guiding mouthguard assembly of the second embodiment of the present invention, the upper teeth-containing channel 201 and the lower teeth-containing channel 202 contain the upper teeth and the lower teeth of the user respectively. As a result, the two upper light-guiding strips 21 are located on the inner side and the outer side of the upper teeth respectively. The two lower light-guiding strips 22 are located on the inner side and the outer side of the lower teeth respectively.

When a lighting device (not shown in the figure) emits light to the ends 210, 220 of the upper light-guiding strips 21 and the lower light-guiding strips 22, the light emitted from the lighting device may be transmitted through the exterior surfaces of the upper light-guiding strips 21 and the lower light-guiding strips 22 to irradiate the inner surfaces and the outer surfaces of the upper teeth and the lower teeth and the mouth tissues at the same time.

Besides, as shown in FIGS. 4 and 5, an air vent 206 is formed through the substrate 203 between the top surface and the bottom surface of the substrate 203. When the user wears the second embodiment of the light-guiding mouthguard assembly of the present invention, the user may inhale and exhale via the air vent 206 to increase the comfort for the user.

What is claimed is:

1. A light-guiding mouthguard assembly comprising:
a light-transmittable mouthguard having a teeth-containing channel formed along a curve as a teeth arch; and
two light-guiding strips located on opposite sides of the teeth-containing channel respectively; wherein
each one of the two light-guiding strips is continuous and uninterrupted and mounted through two opposite terminals of the light-transmittable mouthguard along the teeth-containing channel;
an exterior surface of each one of the two light-guiding strips is light-transmittable.

2. The light-guiding mouthguard as claimed in claim 1, wherein the light-transmittable mouthguard comprises:
a substrate formed along the curve of the teeth arch; and
two light-transmittable walls formed on a top surface of the substrate along the curve of the teeth arch and opposite to each other;
wherein the teeth-containing channel is formed within the substrate and the two light-transmittable walls;
wherein the two light-guiding strips are mounted through the two light-transmittable walls respectively.

3. The light-guiding mouthguard as claimed in claim 1, wherein the light-guiding strips are optical fiber strips.

4. The light-guiding mouthguard as claimed in claim 2, wherein the light-guiding strips are optical fiber strips.

5. The light-guiding mouthguard as claimed in claim 3, wherein each one of the light-guiding strips has an end exposed from the light-transmittable mouthguard.

6. The light-guiding mouthguard as claimed in claim 4, wherein each one of the light-guiding strips has an end exposed from the light-transmittable mouthguard.

7. The light-guiding mouthguard as claimed in claim 5, wherein the light-transmittable mouthguard is an integrally formed mouthguard.

8. The light-guiding mouthguard as claimed in claim 6, wherein the light-transmittable mouthguard is an integrally formed mouthguard.

9. A light-guiding mouthguard assembly comprising:
a light-transmittable mouthguard having an upper teeth-containing channel and a lower teeth-containing channel that are formed along curves as an upper teeth arch and a lower teeth arch respectively;
two upper light-guiding strips located on opposite sides of the upper teeth-containing channel respectively; wherein
each one of the two upper light-guiding strips is continuous and uninterrupted and mounted through two opposite terminals of the light-transmittable mouthguard along the upper teeth-containing channel;
an exterior surface of each one of the two upper light-guiding strips is light-transmittable; and
two lower light-guiding strips located on opposite sides of the lower teeth-containing channel respectively; wherein
each one of the two lower light-guiding strips is continuous and uninterrupted and mounted through the two opposite terminals of the light-transmittable mouthguard along the lower teeth-containing channel;
an exterior surface of each one of the two lower light-guiding strips is light-transmittable.

10. The light-guiding mouthguard as claimed in claim 9, wherein the light-transmittable mouthguard comprises:
a substrate formed along the curves of the upper teeth arch and the lower teeth arch;
two upper light-transmittable walls formed on a top surface of the substrate along the curve of the upper teeth arch and opposite to each other;
wherein the upper teeth-containing channel is formed within the substrate and the two upper light-transmittable walls, and the two upper light-guiding strips are mounted through the two upper light-transmittable walls respectively; and
two lower light-transmittable walls formed on a bottom surface of the substrate along the curve of the lower teeth arch and opposite to each other;
wherein the lower teeth-containing channel is formed within the substrate and the two lower light-transmittable walls, and the two lower light-guiding strips are mounted through the two lower light-transmittable walls respectively.

11. The light-guiding mouthguard as claimed in claim 10, wherein an air vent is formed through the substrate between the top surface and the bottom surface of the substrate.

12. The light-guiding mouthguard as claimed in claim 9, wherein each one of the upper light-guiding strips and the lower light-guiding strips has an end exposed from the light-transmittable mouthguard.

13. The light-guiding mouthguard as claimed in claim 10, wherein each one of the upper light-guiding strips and the lower light-guiding strips has an end exposed from the light-transmittable mouthguard.

14. The light-guiding mouthguard as claimed in claim 11, wherein each one of the upper light-guiding strips and the lower light-guiding strips has an end exposed from the light-transmittable mouthguard.

15. The light-guiding mouthguard as claimed in claim 12, wherein
the upper light-guiding strips and the lower light-guiding strips are optical fiber strips; and
the light-transmittable mouthguard is an integrally formed mouthguard.

16. The light-guiding mouthguard as claimed in claim 13, wherein
the upper light-guiding strips and the lower light-guiding strips are optical fiber strips; and
the light-transmittable mouthguard is an integrally formed mouthguard.

17. The light-guiding mouthguard as claimed in claim 14, wherein
the upper light-guiding strips and the lower light-guiding strips are optical fiber strips; and
the light-transmittable mouthguard is an integrally formed mouthguard.
